(19) United States
(12) Patent Application Publication
(10) Pub. No.: US 2023/0081060 A1
(43) Pub. Date: Mar. 16, 2023

(54) Volumetric Flow Graft and Stents with Near Constant Conductance Flow

(71) Applicant: Seshadri Raju, Jackson, MS (US)

(72) Inventor: Seshadri Raju, Jackson, MS (US)

(21) Appl. No.: 17/817,567

(22) Filed: Aug. 4, 2022

Related U.S. Application Data
(63) Continuation of application No. PCT/US2021/071126, filed on Aug. 5, 2021.
(60) Provisional application No. 63/062,764, filed on Aug. 7, 2020.

(51) Int. Cl. A61F 2/07

(52) U.S. Cl. CPC A61F 2/07; A61F 2240/008

(57) ABSTRACT

The invention includes an improved flow graft. The graft has a portion where the radius r expands with length L over the portion, so that rⁿ/L is a constant in that portion, where n≥4.0, or 1<n<4. It is preferred that the graft has a beginning section that has constant diameter. The invention also includes a method of designing such an expanding graft to suit the application, such as in the venous system, the arterial system, or for dialysis. The invention also includes near constant conductance stents whose radius r can grow with length, so the Rⁿ/L is constant where n>4.

### PRIORITY CLAIM

This application is a continuation of PCT/US/2021/071126, filed Aug. 5, 2021, which application claimed the priority benefit of provisional application No. 63/062,764, filed on Aug. 7, 2020, both of which are incorporated by reference.

### BACKGROUND OF THE INVENTION

Venous grafts or fistulas are tubular members used to move blood from one part of the body to another; for instance, an AV graft or AF fistula used as a shunt or bridge, moving blood from the arterial system to the venous system, such as used in dialysis. A fistula employs natural materials, such as a harvested vein, for the tubular member. For a graft, synthetic non-elastomeric but flexible materials such as plastics (Dacron, polyesters, PVC, polyurethanes, PTFE or Teflon, or eTeflon) are used to form the tubes. Flow rates in these shunts and fistulas are important, as variable flow rates can cause problems in the procedure using the graft. It is desired to have a graft that has improved flow properties, such as constant conductance flow along the length of the graft, for greater flow rates than those present in uniform diameter grafts which exist in the prior art.

### SUMMARY OF THE INVENTION

The invention includes grafts whose radius grows monotonically along the length of the graft. It is preferred that the radius r grows with length L, so that r⁴/L remains constant. The invention includes grafts where the radius grows so that rⁿ/L that remains constant, where n≥4.

The invention includes grafts that grow piecewise linear; for instance, a graft can have a setoff radii rᵢ along the length at positions Li, where each radius and associated length is such that rᵢⁿ/Li is a constant, where n≥4 and the radius between sequential rᵢ grows linearly with length. The grafts can include fitted radial plastic rigid rings at various positions along the length to maintain the shape of the graft. This piecewise growth can also be an approximation of rⁿ growth with length.

### BRIEF DESCRIPTION OF THE FIGURES

FIG. 1 is a cross section through a graft where the radius r grows with length L so that rⁿ/L is a constant.

FIG. 2 is a cross section through a graft that has set radii that grow with length L so that rⁿ/L is a constant, but r is linear with length therebetween.

FIG. 3 is an illustration of a side view of one experimental system used to test the output of an expanding conduit.

FIG. 4A is a set of bar graphs comparing flow rates of constant radius flow versus constant conductance flow in the experimental setup of FIG. 3, through grafts of various radii of length 160 mm where the input pressure was 10 mmHg.

FIG. 4B is a set of bar graphs comparing flow rates of constant radius flow versus constant conductance flow using the experimental setup of FIG. 3, through grafts of various radii of length 160 mm where the input pressure was 25 mmHg.

FIG. 5A is a set of bar graphs comparing flow rates of constant radius flow R versus constant R4/L, R5/L and R6/L flow in the experimental setup of FIG. 3, with and without an air-trap, through grafts of various radii of length 310 mm where the input pressure was 10 mmHg.

FIG. 5B is a set of bar graphs comparing flow rates of constant radius flow R versus constant R4/L, R5/L and R6/L flow in the experimental setup of FIG. 3 through grafts of various radii of length 620 mm where the input pressure was 10 mmHg.

FIG. 6 shows a perspective view of five Z stents assembled in a piecewise construction of a near conductance flow segmented stent.

FIG. 7 shows a near constant conductance flow balloon where a 16 cm portion of the balloon matches the constant conductance flow stent characteristics of the stent it will expand.

### DESCRIPTION OF THE PREFERRED EMBODIMENTS

Blood flow is normally modeled with Poiseuille's law and hydrodynamic relationships, including the expression between flow, pressure, and 1/resistance (or conductance), are as follows:

Poiseuille equation, Fluid flow, Q=ΔP/R where 1/R is the conductance and where R=8 μL/(π⁴), and where μ is the fluid viscosity
Rearranging, and combining the two equations:

Q=ΔP*(π/8)*(r⁴/L)*(1/μ)

where L represents the length of the cylinder (graft) (measured from the start of the graft), the last three terms represent the numeric, geometric, and viscosity factors, respectively. Q, or volumetric fluid velocity (m³/l) or flow, in the venous system, is generally measured in ml/sec or liters/min. These relationships can also be used for modeling volumetric flow. For instance, we constructed a constant conductance flow conduit or “unitary conduit” with an initial diameter of 11.2 mm expanding to an end diameter of 17.7 mm at a length of 6 cm. We compared volumetric fluid flow through this unitary conduit to a second uniform diameter conduit (non-unitary flow). A total of 4.5 liters of fluid with the same viscosity as blood were allowed to run through each conduit with ahead pressure of 25 mm Hg. The time taken to empty the 4.5 liters is shown in Table 1 below.

TABLE 1

| | Non-Unitary | | |
|---|---|---|---|
| | Uniform | | Unitary |
| | Conduit | | Conduit |
| | Time | | Time |
| Trial | (seconds) | Trial | (seconds) |
| 1 | 64 | 1 | 43 |
| 2 | 64 | 2 | 41 |
| 3 | 66 | 3 | 44 |
| 4 | 68 | 4 | 45 |
| 5 | 68 | 5 | 45 |
| Average Time | 66 | Average Time | 43.6 |
| Average Flowrate | 68 mL/sec | Average Flowrate | 103 mL/sec |

Thus, the flow rate for the unitary flow conduit was 103 mL/sec and was 68 mL/sec averaged) for the uniform radius conduit (constant radius). As can be seen, errors in the conduit or graft radius can have significant consequences on blood flow. The flow equations can be simplified further by inserting known values for π and μ (the viscosity of blood). For instance, for a conduit of 1 cm in length with a diameter of 11.3 mm at 1 cm, the formula further reduces to: Q=ΔP*1=ΔP. Such a “unitary conduit” will have a conductance of exactly 1, allowing flow to be directly proportional to the pressure head. This shows the power of expanding the radius of the conduit (the graft). Such a graft, of constant conductance flow (e.g., r⁴/l=constant=K). will be referred to as a unitary graft or a constant conductance flow graft.

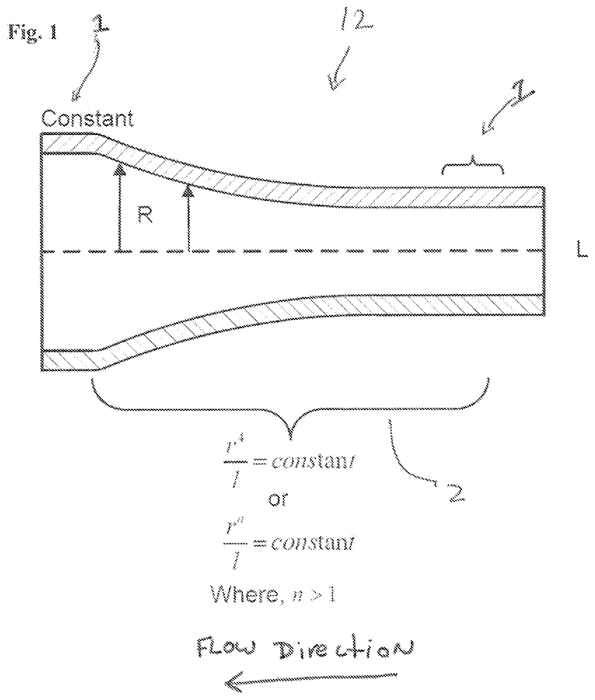

A constant conductance flow graft is particularly useful in grafts that are short in length, typically 20 cm or less. Initial diameters of 6 mm to 14 mm are typically useful in some applications. An example is shown in FIG. 1, which depicts a lengthwise cross section through a graft 12 that starts and ends with sections 1 of constant radius, but grows with rⁿ/l constant tin the section 2 therebetween, where n≥4.

Some uses for grafts follow:

### Dialysis Grafts

Too much flow or too little flow is a problem with grafts in the standard configuration of a uniform diameter graft. Too much flow may result in heart failure, putting too much load on the heart. Too little flow may not clear creatinine, urea, and other substances during dialysis, requiring longer dialysis times or more frequent dialysis. Too little flow also may result in graft thrombosis, a major problem with dialysis grafts at the present time. This requires surgery or interventions to reopen the graft and may ultimately lead to need for a new graft. Some patients run out of places where you can put such grafts in (you need a good artery and vein)—sometimes an indirect cause of fatality.

A low flow situation is suspected to cause a proliferation of fibrous tissue at the site of graft-vein anastomosis in some patients. This proliferation is similar to ISR and is suspected to be the cause of dialysis graft thrombosis in over 80% of the occurrences.

### Artificial Auxiliary Arterio-Venous Fistula

These surgically created artificial fistula are used to increase flow to keep a venous section, bypass, or graft open. Typically, they are used when inflow into a bypass (e.g., Palma femoro-femoral venous bypass) or graft (e.g., iliac vein graft with poor inflow) is considered poor. The A-V fistula may also be used after clearing a clot from a vein (thrombectomy) or scar tissue from a post-thrombotic vein (endophlebectomy). The fistula is meant to be temporary and is closed after a period of 6 weeks or so. Like dialysis grafts, the A-V flow can cause heart failure, but a more common problem is venous hypertension—increased venous pressure in the leg, the correction of which is often the original goal of the primary operation. Using an increased flow graft with a higher rate of flow in the above situations will help prevent thrombosis which may occur from low flow. The variability of flow is worsened where the length of the graft is set by the surgeon, possibly shorten the graft by cutting as desired. A change in graft length creates a change in flow delivered by the graft.

Venous or Arterial Grafts with Poor Inflow or High Rate of Thrombosis

While arterial grafts enjoy high pressure inflow, venous grafts do not because venous pressure is naturally lower. A constant conductance flow graft makes sense in order to preserve the pressure energy from degrading by use of the standard configuration grafts. Examples of such grafts include porta-caval, mesenteric-caval, axillary subclavian, femoro-femoral, femoro-iliac and iliac-caval venous bypasses.

The constant conductance flow grafts may also be useful in short length arterial bypasses where no venous substitute is available. A constant conductance flow graft will also be useful in arterial-arterial grafts where the “run off” or the downstream bed that it flows into is poor.

Grafts with Support

Grafts with rigid skeleton support, such as formed by placing rigid or semi-rigid plastic rings spaced along the graft length and glued to the graft exterior or interior, are useful as endoprosthesis. After deployment, the resident vessel assumes the standard configuration of these devices. They are usually deployed in distressed situations where flow is problematic, and the chance of thrombosis is high. A constant conductance flow configuration makes better sense in these locations.

One such example is a TIPS (trans jugular intrahepatic porta-systemic) shunt performed for portal hypertensions. The device used has a high thrombosis rate from low operating pressures. Using a constant conductance flow graft, employing external rings whose diameter also varies with position, would improve patency.

### Design and Use of a Constant Conductance Flow Graft

The concept is to keep the conductance or flow constant in the graft, which can be achieved by maintaining the geometric factor (r⁴/L) a constant value K in the graft. All examples used herein will have the graft grow after the first 1 cm of length, where the first cm has a constant radius, thus avoiding the ambiguity of examining r⁴/L as L→0. In this instance, the constant K the radius at 1 cm, R1, to the chosen geometric factor, divided by 1 cm, for instance, (R1)⁴/1. In reality, a graft starts in an existing conduit. That implies that the initial conduit/graft combination can be viewed as a single conduit. To determine the “effective length” of that portion of the conduit before the onset of the graft, we have L=ΔPr⁴*π)/(Q*8*μ). Consequently, the combined “conduit” at the beginning of the graft, has a length, and the length of the graft will never be zero. Use of a 1 cm starting length is arbitrary, but not unreasonable, as L should be small. Indeed, L can be estimated using r as the graft radius, the measured Q (such as from doppler sonar), and ΔP, within biological systems other than arterial, should be small, so L will not be very large. Hence, using a 1 cm constant diameter starting graft is arbitrary, but not unreasonable, as it unlikely changes L significantly.

As an example, consider a graft having a diameter of 18 mm (9 mm radius) with a starting length of 1 cm with a constant radius graft, then at the start of the constant conductance flow (at length 1), r⁴/L=(1.8/2)⁴/1=0.6561=K. This number K will be used as the constant K or constant value used in the reminder of the graft. that is, r⁴/L=(1.8/2)⁴/1=0.6561 in the remainder of the graft. Consequently, for a 2 cm long graft, the terminating radius would be (0.6561*2)−^{0 25}=1.070 cm or a diameter of 21.4 mm. A 3 cm long graft would have a terminating radius of (0.6561*3)−^{0 25}=1.18, cm, or diameter of 23.6 mm; a 4 cm long graft would have an ending radius of 12.7 cm, or a diameter of 254 mm; and for a graft length of 5 cm, the terminating radius would be 13.45 cm, or a diameter of 257 mm (an overall increase in cross-sectional area of about 123.3% (1.34/0.9)*2.

As used herein, the “downstream” end of the graft is larger. The downstream direction of flow in the venous system is closer to the heart. Alternatively, downstream is the lower pressure end of the graft. Downstream in the venous system is closer to the heart; in the arterial system, downstream is further from the heart. The “downstream” direction of the graft is the direction that increases in radius. This increase in diameter with length will assist to maintain improved flow in the graft, to prevent graft malfunctions like in-graft restenosis caused by ingrowth of clot/tissue which accumulates and lines the wall of the graft. In addition, as the graft delivers constant conductance flow, the initial upstream end can be a smaller diameter than would be needed in a uniform diameter graft, as a smaller initial diameter constant conductance flow graft can produce the same flow at the outfall or downstream end as that of a uniform diameter graft. We have calculated the length necessary for various diameters in grafts up to 5 cm length, in Table 2. The first cm in length of these grafts is of constant diameter.

TABLE 2

| Graft length | 1 cm | 2 cm | 3 cm | 4 cm | 5 cm |
|---|---|---|---|---|---|
| diameter CIV | 16 | 19 | 21 | 22.6 | 23.9 |
| EIV | 14 | 16.6 | 18.4 | 19.8 | 20.9 |
| CFV | 12 | 14.3 | 15.8 | 17 | 17.9 |

(graft diameter in cm)

As described, for a lengthwise cross section though a graft, the outer envelope preferably creases as a 4^{th} order polynomial with the length. However, in some applications, such as in long grafts, fourth order graft growth (R4 growth or r⁴/l=constant) may present an ending diameter that is too large for the landing site. In this case, a graft that preferably expands monotonically with length, but less than R4 growth, will still provide a benefit, as the flow loss through such a graft will be less than that from R4 growth, but greater than a constant diameter graft. For instance, growth of “near constant conductance flow” is such that rⁿ/n is constant, where n>4, provides such slower growth and flow than R4 growth.

For instance, grafts can be achieved with r⁵/l, r⁶/l or r⁷/l being constant. Faster growth, where 3≥n≥1, in parts of or all of the graft, can also provide benefits, as flow will be greater than that in constant conductance flow, and can be useful in areas of a graft where deposits are a concern.

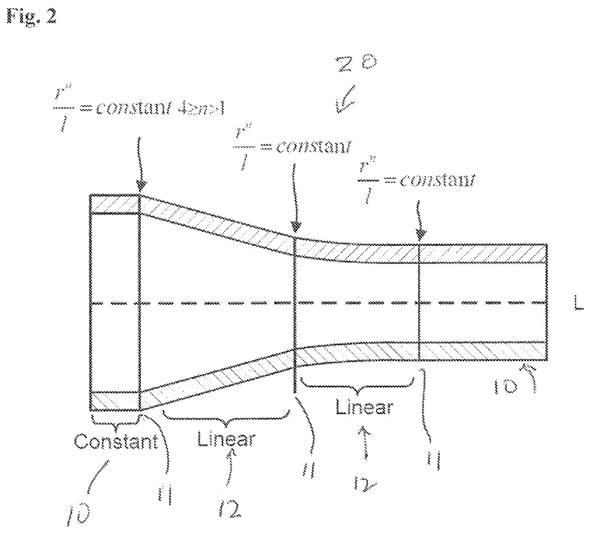

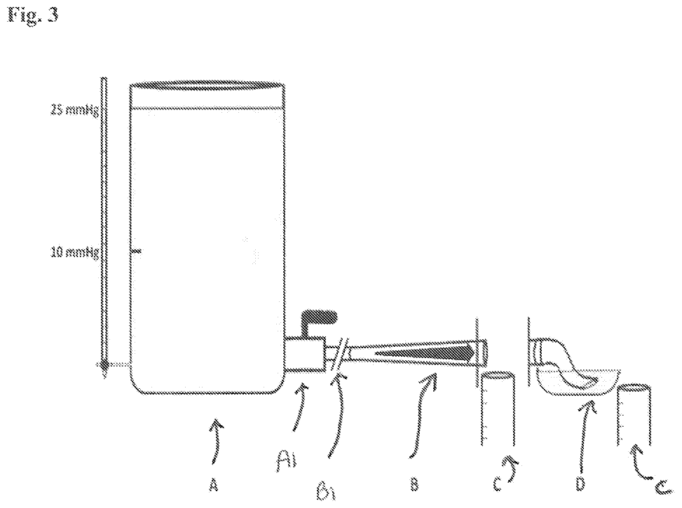

Additionally, growth in the radius with length can occur piecewise. For instance, the graft's outer envelope may linearly increase between set graft radii, ri in piecewise steps. For instance, there could be a series of lengths Li where the radius ri is such that ri⁴/Li=constant. Between such 4^{th} order radii, the graft could grow linearly or by other growth rates. Faster growth, and faster flow, such as linear growth (R1) can be used. Such a step wise growth approximates fourth order growth and may be more efficient to manufacture. However, this is not preferred as the flow will inconsistently change in the graft. While a 4^{th} order polynomial increase (R4) is preferred for the outer envelope of the graft, any preferably monotonically increasing graft diameter with length will provide a benefit, in that radius growth with length helps offset reduced flow with length in the existing constant diameter grafts. An example is depicted in FIG. 2 showing a lengthwise cross section through a graft 20, that starts and ends with constant radius sections 10, and has a series of three radii 11 where amongst themselves, they grow with at rⁿ/l constant, where 1<n≤4 or n≥4, but between the three radii, the growth connecting adjacent radii is linear 12. The set radii of R4 growth could be supported with external or internal rings. Using faster growth rates, such as increasing radius with r³/L or r²/L being constant, (R3 or R2 growth), and connecting those radii with linear growth, is in the scope of the invention Graft growth by x² or x³ or a combination sin whole or in part, can also provide a benefit, as flow is increased, which can be useful in areas of the graft where growth might accumulate from deposits with slower flow. For instance, a portion of the stent can be R4, then change to R3 for a second portion, then revert to R4 to the end.

In one embodiment, for a graft of selected length L1, the upstream and downstream terminating diameters are chosen, and the length and the growth factor Rn are determined to best fit the selected diameters. Alternatively, the outflow and ending diameter are chosen, and the length and growth factor selected to fit the selected parameters. Variations are possible, for a particular ending diameter, a variety of starting diameters, lengths and growths can produce the desired outflow.

Expansion with the chosen growth factor is depicted in FIG. 1. FIG. 2 depicts a cross section through a graft, where the graft's outer envelope approximates R4 growth by having a series of lengths 11 along the graft (shown as three), where r⁴/l is constant. In between these lengths, the growth 12 can be linear. As shown, the ends of the graft 10 have constant diameters sections. This piecewise growth is believed to make fabrication of a stepwise approximation to R4 growth (or other chosen growth factor) more easily constructed but is not preferred as the flow rate will have fairly abrupt changes. The ending radius of each graft segment Si, at length Li (measured from the start of the graft) is preferably unitary, or ri is designed so that ri⁴/Li the same constant value at each length Li. That is, if the length of each segment is li, then the total length of the growth portion Li up to the radius in question, will be Li=Σli, where the sum stops at the segment in question. Each segment's ending radius ri is such that ri⁴/Li is equal to the same constant, ro⁴/Lo, where ro is the radius at the start of the first growth section of the graft (where growth first starts), and Lo is the initial length of the graft to the start of the growth section. In the examples shown herein, Lo is 1 cm in length. While each starting and ending graft segment radius is unitary, the growth of the graft radius between may be either unitary (constant conductance) or unitary like (near constant conductance). For instance, the step grafts in FIG. 2, each starting and ending segment radius 11 is unitary, but the growth within each segment 12, r grows linearly with length. As used herein, Rn or rⁿ growth means rⁿ/l is constant.

In one embodiment, for a graft of selected length L1, the upstream diameter areas well as the growth factor is chosen to provide the desired flows and ending diameter. For a particular ending diameter, a variety of starting diameters, lengths and growths factors can produce the desired outflow. Additionally, the invention includes grafts that have a portion that does not grow, for instance, the starting end of the graft, or the terminating end of the graft can be constant diameter, or both. Preferably the graft diameter is monotonically increasing over the length of the graft. Typical starting diameters can be 4 mm, 6 mm or 8 mm such as, for instance, dialysis. The smaller diameters are useful when tapping small veins.

Graft Growth with Length

Flow, as used herein, is fluid velocity. For instance, the growth of each graft segment in FIGS. 1 and 2 are not necessarily unitary R4 growth or constant conductance flow grafts (but can be) between the starting and ending segment diameters, but can be linear, or polynomial growth, r, r⁵, r⁶ or r⁷, squared growth, or r³ growth, or a combination. Grafts that grow in diameter constantly, or monotonically, not by the 4^{th} degree, but rⁿ growth such as a first (x), second (x²) or third degree (x³) (1≤1n<4) or fifth degree (r³) sixth degree (r⁶), seventh degree (r⁷) growth, or higher (n>4) or combinations thereof are considered to be “unitary-like” or “near constant conductance flow” grafts. Where rⁿ/l is constant, n<4 implies faster radial growth and flow greater than constant conductance R4 flow, and where n>4, implies slower radial growth and flow than constant conductance R4 flow. Note. When growth of r with L is less than R4, you still get a benefit, as the resultant flow is greater than that of a constant diameter graft.

Radius growth with length is within the scope of the invention. All such will provide a benefit, as the increasing graft diameter helps offset reduced flow with length. For instance, increasing radii with rⁿ/L constant, where n>4 is constant overall or at specified intervals, and connecting those intervals linearly as shown in the graft in FIG. 2, is in the scope of the invention, as is constant expansion with the chosen geometric factor, as shown in FIG. 1. Slower growth rates, e.g., rⁿ/L is constant, where n>4, such as n=5, 6, or 7 e.g., R5, R6, or R7 growth, or higher, will be beneficial where long length grafts are needed, providing for slower growth than R4. Hence the flow is less; the benefit is that the ending radius size is less than that in R4 growth, and therefore is more likely to be acceptable in a biological conduit system, such as a vein or artery in long length grafts, but still providing for improved flow over that in constant diameter graphs.

For instance, fabrication of a graft with R4 growth will yield a gradually expanding tube that will double its radius at 16 cm length. In many applications, this growth is too fast, resulting in an ending radius that is too large for the application. A more practical formulation is to keep r⁵/l or even r⁶/l or r⁷/l constant over the length of the conduit after the first cm. This will yield longer tube lengths before the radius doubles (Table 3); the conductive performance (flow) will be less than the R4 growth, but still with improved flow, greater than that of existing uniform cylindrical grafts. The fractional constants necessary for the various integer radius exponents vs length are shown in Table 4 for a number of selected initial calibers. Note, n does not have to be an integer.

TABLE 3
Conduit radius increase with length

| | Length at |
|---|---|
| Constant | which Rᵢₙᵢₜᵢₐₗ |
| Variable | doubles (cm) |
| $\frac{{r}^{2}}{L}$ | 4 |
| $\frac{{r}^{3}}{L}$ | 8 |
| $\frac{{r}^{4}}{L}$ | 16 |
| $\frac{{r}^{5}}{L}$ | 32 |
| $\frac{{r}^{6}}{L}$ | 64 |

TABLE 4
Initial and end diameter of uniform cylindrical and test conduits

| | | Ending | Ending | |
|---|---|---|---|---|
| Constant | Initial | Diameter at | Diameter at | Ending Diameter |
| Geometric | Diameter | L = 160 mm | L = 310 mm | at L= 620 mm |
| Factor | (mm) | (mm) | (mm) | (mm) |
| r | 4 | 4.00 | 4.00 | 4.00 |
| | 6 | 6.00 | 6.00 | 6.00 |
| | 8 | 8.00 | 8.00 | 8.00 |
| | 10 | 10.00 | 10.00 | 10.00 |
| $\frac{{r}^{4}}{L}$ | 4 6 | 8.00 12.00 | 9.44 14.16 | 11.22 16.84 |
| | 8 | 16.00 | 18.88 | 22.45 |
| | 10 | 20.00 | 23.60 | 28.10 |
| $\frac{{r}^{5}}{L}$ | 4 6 | 6.96 10.45 | 7.95 11.92 | 9.13 13.70 |
| | 8 | 13.93 | 15.90 | 18.26 |
| | 10 | 17.41 | 19.87 | 22.83 |
| $\frac{{r}^{6}}{L}$ | 4 6 | 6.35 9.52 | 7.09 10.63 | 7.96 11.94 |
| | 8 | 12.70 | 14.18 | 15.92 |
| | 10 | 15.87 | 17.72 | 19.89 |

To demonstrate performance of slower growth grafts, the following experiment was done:

### Fabrication of Experimental Conduits

To test less aggressive growth graft designs, r° growth conduits, n>4. were designed using engineering software (Autodesk, Inc.; San Rafael, Calif.) and fabricated in a commercial 3D printer (Stratasys; Eden Prairie, Minn.).

### Experimental Test Model

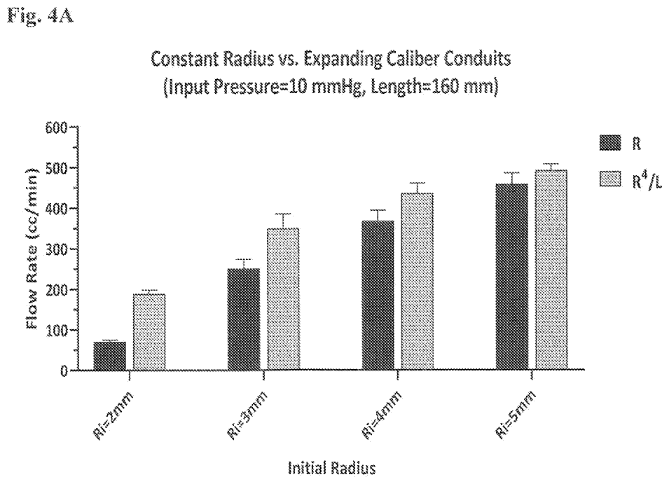

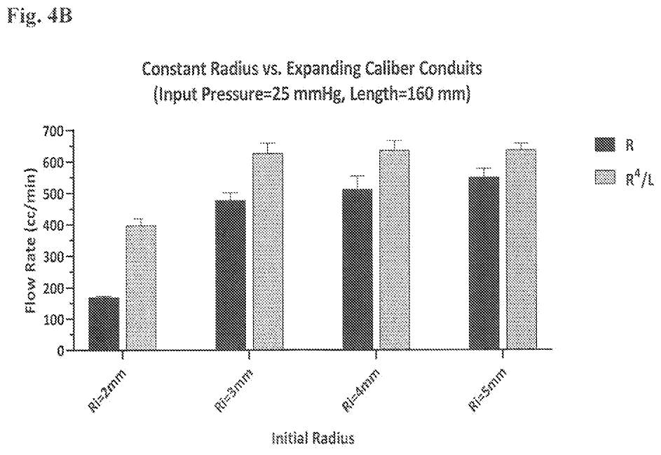

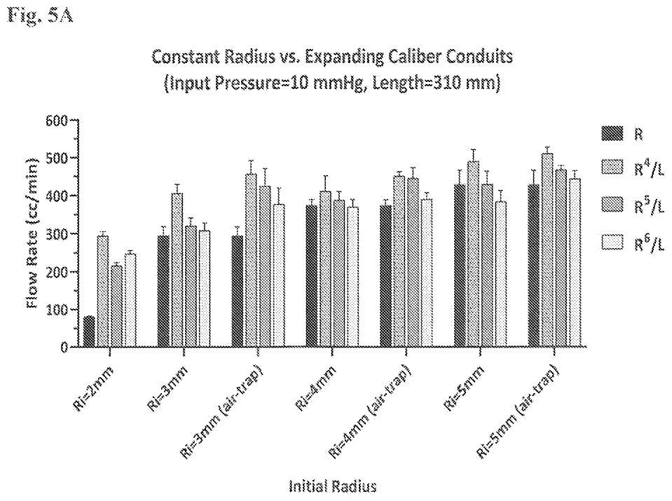

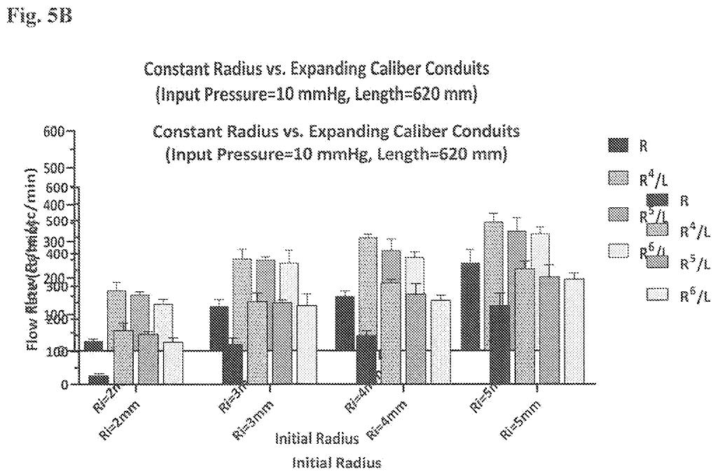

The basic flow model consisted is of a header tank A with outflow controlled by a calibrated ball valve A1 (FIG. 3). The ball valve A1 was kept open at the same setting for all flows. The various conduits B tested were connected to the ball valve A1. Each conduit B had an initial starting length, B1 (here, 1 cm), wherein the radius was constant. It is believed that the constant starting radius B1 allows flow from the pressurized system to stabilize before entering the expanding section. It also avoids the potential ambiguity of evaluating rⁿ/l as 1→0. Conduit outflow was open to the atmosphere (open system) and was allowed to drain into a graduated cylinder C for timed measurements (cc/minute). In some experiments, a partially closed system of drainage was used: the conduits B were connected to a short Penrose drain D (Diameter=3.5 cm; Length=13 cm) discharging under the fluid level in a shallow pan before emptying into the output cylinder C (optional). The system prevented air from entering the conduit at the discharge end, functioning similar to the Heimlich valve. The tank system was filled with a 2:3 mixture of glycerol and water with a viscosity of 0.04 poise. Each flow measurement is an average of 5 runs or trials. As shown in the bar graphs of FIGS. 4A and 4B, the flow rate of a constant diameter conduit is always less than the expanding conduits, for all length tested (1.6 cm, 3.1 cm and 6.2 cm) and for all starting radii tested of 2 mm, 3 mm 4 mm, and 5 mm. As expected, flow rates with r=⁴√l were greater than flows with r×⁵√l constant, and which flows were greater than flows than r=⁶√l constant, for two different starting pressures. See FIGS. 5A and 5B.

### Results

The flow rates of expanding caliber conduits (r⁴⁻⁶) compared to traditional constant radius cylindrical conduits are shown in Table 5 and FIGS. 5A and 5B. The expanded caliber yields a significantly improved flow from 14% to 563% in all but a few instances. In the latter instances, the outflow stream was observed to be separated from part of the tube outlet circumference, suggesting flow separation from the wall (i.e., the flow was no longer laminar for some length near the outflow end). This problem was substantially reduced when the Penrose air-trap was used (Table 6). The air trap restricts the flow of air into the conduit at the discharge end, more closely simulating a closed fluid flow system, like the arterial or venous system, where flow separation/cavitation should not be an issue,

In closed flows where the fluid completely fills the conduit and the flow is driven by a pressure gradient, the incidence of transition to non-laminar flow should be reduced, as in an expanding pipe, where rⁿ/l is constant and where n≥4, the fluid velocity declines with length, reducing the potential for turbulence.

### Designs

Accretive manufacturing (3-D printing) makes it much easier to fabricate expanding caliber grafts for biological use. There is a practical limit to the length of the graft depending upon location and use. Examination of Table 3 (length of conduit when initial radius doubles) and Table 5 (measured flow rated for the conduits tested without an air trap) suggests that up to ˜16 cm is practical for graft designs keeping r⁴/L constant. As shown in Table 6, for common iliac vein grafts of 14 mm diameter, the ending radius is calculated for various length conduits for different r^{N} N=4, 5, or 6. with all conduits having an initial starting length (1 cm) of constant radius. As shown, combinations up to 64 cm length appear practical for grafts keeping r⁵/L constant. Longer lengths may be required for particular applications and are possible keeping r⁶/L or r⁷/L or higher r values constant. Fabrication techniques described above or known to those of ordinary skill in the art may be used to construct the Rn expanding stent/conduit.

TABLE 5
Mean conduit flow rate when R, R⁴/L, R⁵/L, and R⁶/L
are held constant (no air-trap)

| | | | | | |
|---|---|---|---|---|---|
| | | | Constant | Constant | Constant |
| | | | R⁴/L | R⁵/L | R⁶/L |
| | | Constant | Flow (cc/ | Flow (cc/ | Flow (cc/ |
| Conduit | Initial | Radius | min) (% | min) (% | min) (% |
| Length | Radius | Flow | improve- | improve- | improve- |
| (mm) | (mm) | (cc/min) | ment) | ment) | ment) |

Input Pressure = 10 mmHg

| | | | | | |
|---|---|---|---|---|---|
| 160 | 2 | 71 | 188 | — | — |
| | | | (+165%***) | | |
| | 3 | 251 | 349 | — | — |
| | | | (+39%***) | | |
| | 4 | 368 | 435 | — | — |
| | | | (+18%**) | | |
| | 5 | 458 | 492 (+7%) | — | — |
| 310 | 2 | 81 | 294 | 216 | 247 |
| | | | (+263%***) | (+167%***) | (+205%***) |
| | 3 | 294 | 406 | 321 (+9%) | 308 (+5%) |
| | | | (+38%***) | | |
| | 4 | 373 | 411 (+10%) | 387 (+4%) | 369 (−1%) |
| | 5 | 428 | 489 (+14%*) | 428 (0%) | 383 (−11%) |
| 620 | 2 | 26 | 166 | 154 | 129 |
| | | | (+538%***) | (+492%***) | (+396%***) |
| | 3 | 122 | 253 | 249 | 240 |
| | | | (+107%***) | (+104%***) | (+97%***) |
| | 4 | 149 | 310 | 275 | 256 |
| | | | (+108%***) | (+85%***) | (+72%***) |
| | 5 | 240 | 352 | 327 | 320 |
| | | | (+47%***) | (+36%**) | (+33%**) |

Input Pressure = 25 mmHg

| | | | | | |
|---|---|---|---|---|---|
| 160 | 2 | 169 | 398 | — | — |
| | | | (+136%***) | | |
| | 3 | 478 | 628 | — | — |
| | | | (+31%***) | | |
| | 4 | 513 | 636 | — | — |
| | | | (+24%***) | | |
| | 5 | 550 | 637 | — | — |
| | | | (+16%***) | | |
| 310 | 2 | 157 | 427 | 301 | 285 |
| | | | (+172%***) | (+92%***) | (+82%***) |
| | 3 | 401 | 512 | 377 (−6%) | 386 (−4%) |
| | | | (+28%***) | | |
| | 4 | 475 | 575 | 447 (−6%) | 487 (+3%) |
| | | | (+21%***) | | |
| | 5 | 549 | 662 | 491 | 520 (−5%) |
| | | | (+21%**) | (−11%*) | |
| 620 | 2 | 68 | 451 | 364 | 301 |
| | | | (+563%***) | (+435%***) | (+343%***) |
| | 3 | 267 | 520 | 476 | 433 |
| | | | (+95%***) | (+78%***) | (+62%***) |
| | 4 | 403 | 551 | 504 | 509 |
| | | | (+37%***) | (+25%***) | (+26%***) |
| | 5 | 503 | 632 | 592 | 522 (+4%) |
| | | | (+26%**) | (+18%***) | |

*P < 0.05 vs. constant radius flow
**P < 0.01 vs. constant radius flow
***P < 0.001 vs. constant radius flow

TABLE 6
Mean conduit flow rate with and without Penrose air-trap (conduit length = 310 mm)

| | | | | | | | |
|---|---|---|---|---|---|---|---|
| | Constant | Constant | Constant | Constant | Constant | Constant | Constant |
| Initial | R | R⁴/L | R⁴/L + air-trap | R⁵/L | R⁵/L + air-trap | R⁶/L | R⁶/L + air-trap |
| Radius | Flow^{a} | Flow | Flow | Flow | Flow | Flow | Flow |
| (mm) | (cc/min) | (cc/min, %) | (cc/min, %) | (cc/min, %) | (cc/min, %) | (cc/min, %) | (cc/min, %) |

Input Pressure = 10 mmHg

| | | | | | | | |
|---|---|---|---|---|---|---|---|
| 3 | 294 | 406 (+38%***) | 456 (+55%***) | 321 (+9%) | 424 (+44%***) | 308 (+5%) | 376 (+28%**) |
| 4 | 373 | 411 (+10%) | 450 (+21%***) | 387 (+4%) | 444 (+19%***) | 369 (−1%) | 389 (+4%) |
| 5 | 428 | 489 (+14%*) | 500 (+17%*) | 428 (0%) | 467 (+9%*) | 383 (−11%) | 443 (+4%) |

Input Pressure = 25 mmHg

| | | | | | | | |
|---|---|---|---|---|---|---|---|
| 3 | 401 | 512 (+28%***) | 570 (+42%***) | 377 (−6%) | 553 (+38%***) | 386 (−4%) | 449 (+12%*) |
| 4 | 475 | 575 (+21%***) | 602 (+27%**) | 447 (−6%) | 566 (+19%**) | 487 (+3%) | 505 (+6%) |
| 5 | 549 | 662 (+21%**) | 638 (+16%**) | 491 (−11%*) | 602 (+10%**) | 520 (−5%) | 553 (+1%) |

*P < 0.05 vs. constant radius flow
**P < 0.01 vs. constant radius flow
***P < 0.001 vs. constant radius flow
^{a}Flow separation did not occur in the constant radius conduits; Penrose air-traps did not affect these flows.

The Rn expanding caliber grafts may have an advantage over the traditional cylindrical prosthetics in the following areas of vascular surgery:

### Dialysis Grafts

Inadequate flow for effective dialysis is a common problem with legacy dialysis grafts. A 3 mm radius 31 cm length conduit (which approximates commonly used dialysis grafts) yields 294 and 401 cc/min (10 &25 mm Hg input pressure) in the test bed. In comparison, the expanding configurations yield a 28-38% more flow (nearly 50% more flow with the Penrose air-trap). These flows are for 10- and 25-mm Hg input pressures, respectively. Quantitative flows in patients may be different for higher input pressures. While quantitative duplication of clinical flows in the test bed is not to be expected, the relative flow advantage is a useful indicator. Graft thrombosis and intimal hyperplasia likely will be reduced. Grafts are used to correct intimal hyperplasia at the venous end of dialysis grafts and fistulas. An expanding caliber graft may function better in these locations. Grafts are also used to correct intimal hyperplasia at the venous end of dialysis grafts and fistulas.

### Venous Grafts

Venous grafts were used in veno-venous bypasses 50 years ago but have largely fallen out of use since the introduction of venous stents. Even then, it was known that grafts to be used in the venous system had to be larger (lesser resistance) than the ones used in the arterial system, as the pressure gradient is lower in the venous system. Prosthetic grafts are still occasionally required in complex reconstructions involving the large central veins. The expanded configuration may function better in these situations. Grafts are also used to correct intimal hyperplasia at the venous end of dialysis grafts and fistulas. An expanding caliber graft may function better in these locations.

### Arterial Grafts

Prosthetic use in arterial applications has declined as well, substituted by stents or autogenous material. Arterial grafts are still used to a greater extent in arterial bypasses than in the venous system. The expanded configuration is likely more efficient and may have improved long-term patency than the legacy design in these applications. Prosthetic grafts are avoided in peripheral arterial bypasses in general and particularly when the inflow or outflow is marginal reducing the pressure gradient to maintain flow. The expanded configuration may find a place for use in these challenging situations. Use of prosthetics is faster (less operating time) and less laborious than harvesting or constructing autogenous grafts from autologous material (which may be unavailable).

### Stent Supported Grafts

These composite stent/grafts are used in specific anatomic locations where the prosthetic is subject to external compression/stress. Flow characteristics of the expanded configuration may function better where both the stent and graft expand equally. Indeed, the stent expansion may be greater, but the graft will control/limit the expansion of the stent. Both should be expanding in diameter with length.

As will be understood by one of skill in the art, the length in in rⁿ/l in constant conductance flow grafts (r=⁴√l), or the near constant conductance flow conduits, is measured from the start of the conduit, not the start of the expanding section. This provides for smooth transition of flow through into the expanding section. Conduits (r=ⁿ√l where n>4) can have endless applications where the flow rate through the application is an important factor to the functioning of the system, including the arterial system. In biological systems, use of expanding radii grafts should greatly reduce restenosis in these grafts.

### Designs

In one embodiment, for a graft of selected length L1, the upstream and downstream terminating diameters are chosen (for instance, the upstream diameter>4 for slower growth and slower flow rates than a constant flow design. If the resulting outflow is too fast, a short ending segment might have a constant diameter, or a reducing diameter section, to slow the flow. Alternatively, the starting and ending diameters can be chosen, the selected length, and then solve for the best filling value of n in rⁿ/l, over the selected length of non-constant section. Note that n does not have to be an integer value. A similar design can be utilized to construct long grafts in steps or segments, by choosing the segment ending diameters and selecting the geometric expansion to be used between the segments.

Grafts can have one or more portions that are constant conductance flow or near constant conductance flow. Consider a graft that has an initial radius r1 of 15 mm (or 1.5 cm) and remains constant for two cm. For the next 5 cm, the graft is unitary, with the starting radius of 1.5 cm. In other words, for the next 5 cm, r⁴/L remains constant, where L is the distance from the starting point of the graft, (here L starts at 2 cm), that is, to the 7^{th} cm of the graft, L=7 cm. At the end or the unitary graft (or near unitary) portion, the radius in the final portion may further increase, remain constant or even decrease (not preferred). For instance, at the end of the unitary portion described above, the graft may continue for another 2 cm but over that 2 cm, the radius may smoothly decline, such as linearly (e.g., a first order polynomial), to end at the normal radius of the resident vein, and thus allow for a smooth flow transition from the end of the unitary portion to the end of the graft.

### Venous Stents

The considerations above for long grafts, that is, having a portion of the graft with near constant conductance flow, are also applicable to long venous stents, or stent stacks (consecutive stents placed end to end possibly with overlap).

A stenosis is often treated with a stent. Stents are generally cylindrically shaped devices which function to expand when deployed. Stents may be balloon expandable or self-expanding. The balloon expandable stent is a stent that is usually made of a coil, mesh, or zigzag design. The stent is pre-mounted on a balloon and the inflation of the balloon plastically expands the stent with respect to the balloon diameter. Self-expanding stents are tubular devices stored in an elongated configuration in what is called a delivery system or applicator. The applicator is introduced percutaneously into the body into a vessel and guided through the vessel lumen to the location where the stent is to be released. Upon release, the stent material auto expands to a predetermined size. Auto expansion is rather weak in many self-expanding stents. This may require pre-dilatation of the stenotic lesion with a balloon of appropriate size before the stent is deployed to enable it to expand to its intended size. In some stents, auto expansion must be assisted with ‘post dilatation’ for full expansion of the stent to occur.

Commonly used self-expanding stents are braided stents, or laser cut stents. A braided stent is a metal stent that is produced by what is called a plain weaving technique. It is composed of a hollow body, which can stretch in the longitudinal direction and whose jacket is a braid made up of a multiplicity of filament-like elements which, in the expanded state of the braided stent, intersects a plane perpendicular to the longitudinal direction at a braid angle. Laser cut stents are constructed from a tube of material (most frequently, nitinol, a nickel titanium alloy), and stainless steel, cobalt, etc. that is laser-cut during production to create a meshed device. The tube is comprised of sequential aligned annular rings that are interconnected in a helical fashion. The tube is compressed and loaded into the delivery device and expands to original size when released. Nitinol, which has thermal memory, may help stents made of this material expand into position when exposed to body temperature after delivery. Compared with self-expanding braided stents, laser cut stents provide more accurate stent deployment with less foreshortening. Laser cut stents are much less subject to foreshortening but are probably less rigid than braided stents.

The stent, after expansion, is intended to restore the occluded vessel to normal or near normal flow conditions in the stented area. In the arterial and venous system, the stented area should have smooth laminar blood flow of uniform velocity. To help avoid restenosis, or the depositing of material in the stented vein, and the resultant re-occurrence of an occlusion, maintaining adequate flow through the stent is desirable. A stent with a growth portion that is placed over the stenotic area should alleviate these issues.

The iliac veins are the most common location for stent placement. The common femoral vein is ≈12 mm in diameter, the external iliac vein is ≈14 mm in diameter; and the common iliac vein is slightly larger at ≈16 mm diameter (venous caliber naturally scales up as tributaries coalesce). A gradual increasing growth configuration will likely provide greater flow than current constant radius cylindrical designs. In-stent restenosis is a substantial problem in iliac vein stents and correlates with low inflow. It is believed that the expanding stent will ameliorate these problems of legacy designs. The most frequent cause of stent thrombosis is poor inflow; outflow problems are less frequent causes. In either case, the pressure gradient (ΔP) is reduced, causing flow decrease. A greater volumetric flow rates may be possible with a reduced pressure gradient if the expanded configuration stent is used.

In the iliac system, a stent conduit extending from the common femoral vein (12 mm diameter) to the external iliac (14 mm diameter), to the common iliac (16 mm diameter) can be as much as 18 or 19 cm long. As seen in Table 3, at R5 growth, the stent or stent stack through the iliac veins, starting at the common femoral vein, would double to 24 mm at 32 cm. Hence R5, R6 or R7 stent growth could easily be used for stenting the entire iliac vein system. A first stent in the stack would have a starting diameter of 12 cm, and overlapped with the second stent, would have an ending diameter of about 14 cm. Given the length to be covered by the first stent, solve for the best fitting value of N. Repeat for the second and third stents in the stack, the solved for N for each will likely not be the same. Note that N in RN growth does not have to be an integer. Such long stents or stent stacks may jail the hypogastric vein, which is well tolerated.

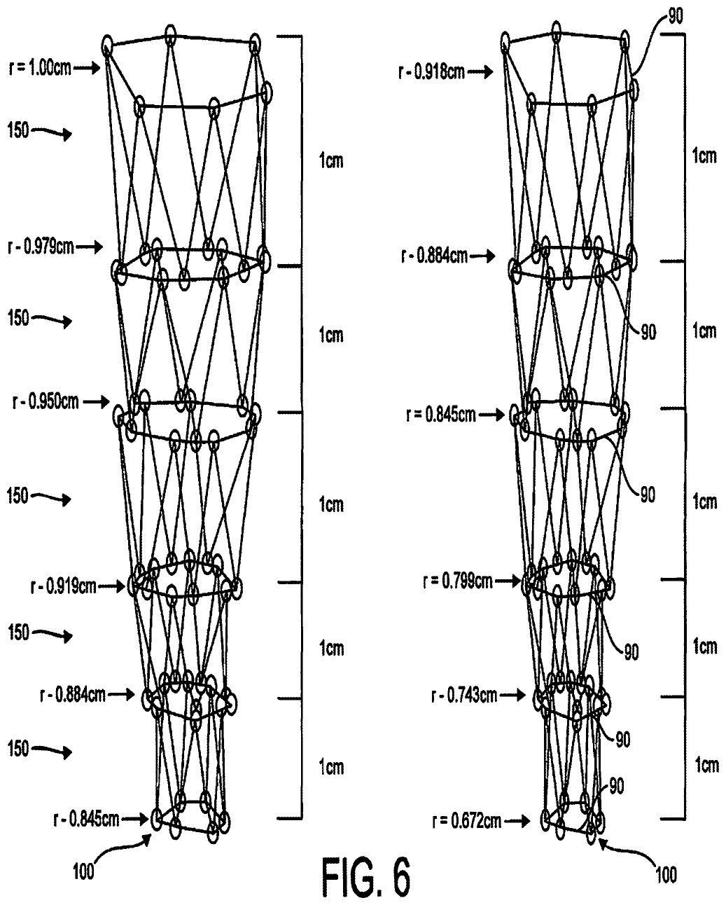

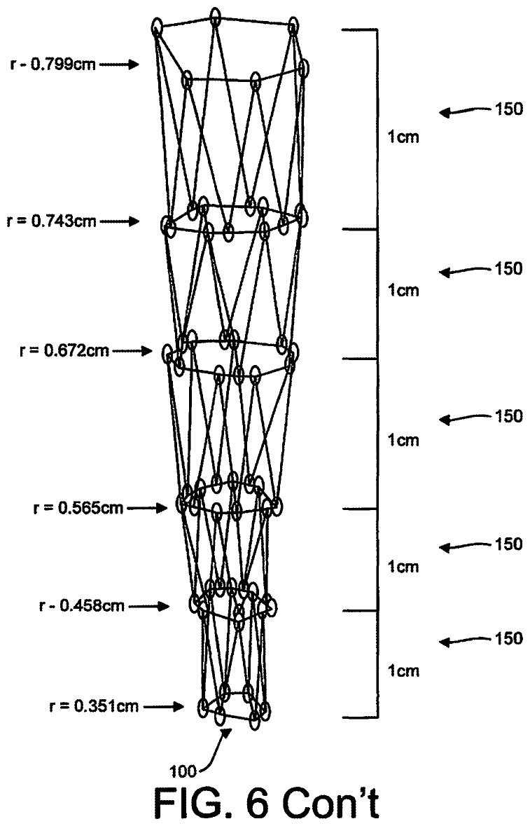

As with grafts, stents can be constructed which are piecewise approximation of stents of R5 and lesser growth, where certain radius are sized to fit rⁿ/L constant, n>4, and these radii connected by linear radius growth between adjacent radii. This linear approximation can be readily constructed with existing Z stents, as is shown in FIG. 6, which shows three Z stent stacks 100, where each stack is constructed for five Z modules or stents 150, where the radius growth between the encircling sutures 90 is linear by design of the Z struts. Suture length can be chosen to produce RN growth. at the fixed radii 90 at the bottom top and in the middle of a two stack Z stent. Z stents are described in U.S. Pat. No. 5,282,824, incorporated by reference.

Indeed, longer stents can be constructed with sections of different growth, such as constant radius (no growth) sections to an R5 growth or higher section and ending with a no growth or R4 growth section. The preferred growth pattern is monotonic growth (non-decreasing growth) over the length of the stent or graft. This piecewise approximation in RN growth may make manufacturing easier, for instance, building a long stent using sections of Z stents.

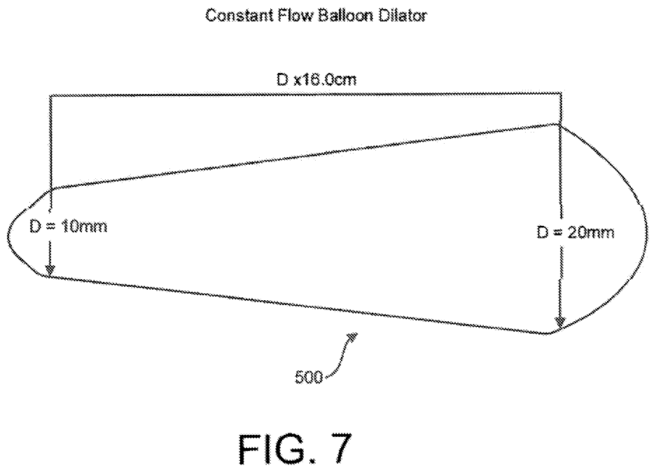

Stents (r=ⁿ√l where n>4) can have endless applications where the flow rate through the application is an important factor to the functioning of the system, including the arterial system. Stents are also used to correct intimal hyperplasia at the venous end of dialysis grafts and fistulas. An expanding caliber stent may function better in these locations. In biological systems, use of expanding radii stents should greatly reduce restenosis in these stents. Note for stents—stent balloons 500 are needed that, when expanded, match the form of the expanded stent. See FIG. 7. Such a balloon can be constructed by placing a graft or sleeve over the balloon (not shown), where the graft or sleeve has the desired radius growth shape. As the balloon is expanded, the graft will guide and limit the expanded balloon into the desired shape. A gradual increasing radius growth configuration stent will provide greater flow than current constant radius cylindrical designs.

### Measurement of L in a Growth Section

As described, it is preferred that in a growth section where R^{N}/L is constant, that L is measured from the beginning of the conduit system (graft or stent). If you measure the length from the start of the growth conduit, then the growth in this case is not identical to that when length is measured from the start of the system. This occurs because r=ⁿ√(K1) in the growth section. The radius is smaller in a growth section when L is measured from the start of the conduit system. Note also that the growth constant K is a different value in the two systems, as K=(rs)ⁿ/Ls, where rs is the radius at the start of the growth section, and Ls is the conduit length at the start of the growth section.

As an example, consider a two-conduit system, each 10 cm length, with a 1 cm overlap, where the first conduit is constant, radius of 2 cm, the second conduit grows at R4 after the 1 cm overlap.

| L = 0 at beginning of system | L = 0 at start of growth conduit |
|---|---|
| K = 2⁴/10 = 1.6 | K = 2⁴/1 = 16 |
| length at end of | length at end of |
| second conduit = 19 | second conduit = 10 |
| (radius at L = 19) = | radius at L = 10) ) = |
| ⁴\sqrt{KL} = ⁴\sqrt{1.6 * 19} = 2.34 | ⁴\sqrt{KL} = ⁴\sqrt{16 * 10} = 3.55 |

Clearly, the two measurements of L result in a different growth profile.

Measuring L in a growth section from the start of the conduit in a growth section is more manufacturer friendly. Otherwise, the manufacturer will have to custom build each conduit, with an understanding of the length of the conduit system prior to the conduit in question. Measuring L from the start of the conduit system more closely emulates a single conduit system, particularly in performance.

You can build a conduit system using growth conduit sections where the growth is referenced from the start of the growth conduit. Such a conduit system will have different growth profile and different performance characteristics than one where length L is measured from the system start. Care should be taken understanding length measurement which system was used.

As can be seen, Constant conductance and near constant conductance flow grafts and stents allow the surgeon to design a graft and/or stent with suitable starting and ending diameters, with an expanding portion in the graft or stent therebetween, providing for improved flow grafts or stents. The variations are almost limitless, particularly as n in rⁿ does not have to be an integer value. Indeed, if n is an integer, rⁿ can also represent a n^{th} order polynomial. A graft or stent can be constructed with two or more portions, each portion expanding, but with possibly different growth factors; or, if the final flow is too fast, a final section may have a greater decreasing diameter section to produce the desire outfall or outflow, but with possibly greater growth factors, such as r2 or r3. It will be apparent to those skill in the art that here are other biological applications for the improved flow graft and long stents.

1. A improved flow graft of length L comprising a structure having a tubular shape when expanded, where the expanded shape includes a radius r at each length l of the graft, such that when expanded, the graft has a portion where the radius r at each point of the length in the portion, is such that rⁿ/l is a constant at each point in the portion, where n=4, or where the growth of the radius r at each point of the length in the portion is monotone with the length in the portion, but where the growth of r with length is less rapid then that of r⁴/l=constant, where the length l is measured from a beginning of the graft.

2. The improved flow graft of claim 1 where r expands with length in the portion such that that rⁿ/l is a constant at each point in the portion, where n is constant, and n≥4.

3. The improved flow graft of claim 1 where the portion terminates at an end of the graft.

4. The improved flow graft of claim 1 where the portion begins after a beginning end of the graft and ends before a termination of the graft.

5. The improved flow graft of claim 2, where the radius at the start of the expanding portion is R0 at length L0, and the constant is r0ⁿ/L0 where n is the selected n in rⁿ/l is constant.

6. The improved flow graft of claim 1 such that r⁴/l is a constant at each point in the portion.

7. The improved flow graft of claim 1 having a series of plastic rings attached to the graft at a series of lengths Li, where the dimeter of each plastic rings matches the diameter of the graft at each location.

8. An improved flow graft comprising a series of tubular segments Si, each segment Si having a radius r at each length L for each segment, each segment having a starting radius and an ending radius such that each starting radius and ending radius is such at rⁿ/L is constant at the starting and ending radius, where n≥4.0 or 1<n<4 and where the radius between the starting and ending radius grows monotonically with length in the segment.

9. The improved flow graft of claim 8 where the growth between the starting and ending radius is linear.

10. A method of designing a graft, where the graft has a radius r at each length L of the graft, comprising the steps of choosing a starting radius rs of the graft and a desired ending radius re of the graft, where re>rs, selecting a length L of the graft, set the radius of the graft constant for a first portion of the graft, and selecting an N such that N≥4.0 so that r^{N}/L is a constant from the end of the first portion, to the end of the graft, and where the ending radius of the graft is the best approximation to re.

11. The graft of claim 1 where the graft is a dialysis graft.

12. The graft of claim 1 where the graft is a venous graft.

13. The graft of claim 1 where the graft is an arterial graft.

14. The method of claim 10, where the ending diameter of graft is designed to be positioned downstream in a biological flow.

15. An expanding sleeve system comprising a tubular shaped non-elastic sleeve, the sleeve having a radius r at each point of a length l of the sleeve, and the shape further comprising a portion where rₙ/l is a constant in that portion, where n>4.0.
